(19) United States
(12) Patent Application Publication
(10) Pub. No.: US 2002/0164361 A9
(43) Pub. Date: Nov. 7, 2002

(54) Cosmetic compositions comprising at least one polyvinyl alcohol, and processes for preparing said compositions

(76) Inventor: Florence L'Alloret, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/809,299

(22) Filed: Mar. 16, 2001

(30) Foreign Application Priority Data
Mar. 16, 2000 (FR) 00 03396

(51) Int. Cl. A61K 7/04; A61K 31/765

(52) U.S. Cl. 424/401; 424/78.17

(57) ABSTRACT

Cosmetic compositions, such as solid aqueous cosmetic compositions, such as solid aqueous gels, comprising, in a cosmetically acceptable medium, at least one polyvinyl alcohol, wherein said compositions have a stress measurement ranging from 200 to 10,000 Pascals. Processes for preparing such compositions comprising (1) cooling a homogeneous aqueous solution of at least one polyvinyl alcohol to a temperature of less than or equal to −5° C., and (2) heating said solution to a temperature of 20-25° C.

The present invention relates to cosmetic compositions, such as solid aqueous cosmetic compositions, such as care and make-up compositions, for example, solid aqueous gels, that can be for example in the form of a stick or a tube.

Many products which are in solid form are known in the cosmetics industry. Mention may be made of, for example, in the make-up field, tubes or “sticks” of lipstick, foundation or eyeshadow; in the field of skincare or lip care, lip-repairing pencils and depigmenting, make-up-removing or moisturizing tubes or “sticks”; in the field of hygiene, deodorant sticks and foaming sticks or bars for shaving or washing the skin. It is desirable to have available products in solid form, because products in the form of solids tend to be practical to use and can be easy to transport, as there is little to no risk of the product running.

These products are generally formulated on the basis, on the one hand, of a fatty phase, which may provide comfort and/or softness, and, on the other hand, of a pulverulent phase, which comprises the desired color of the make-up products. This pulverulent phase may comprise at least one additive chosen from, for example, pigments, fillers, and nacres. The fatty phase generally comprises at least one compound chosen from, for example, waxes, oils, and pasty compounds. However, sticks formulated with a wax base may have certain drawbacks: a greasy nature, which may not be appreciated by all users, and a lack of freshness when applied. In addition, it is difficult to introduce hydrophilic active agents therein.

There is therefore an increasing trend toward cosmetic sticks comprising an aqueous phase in a higher concentration.

It is known practice to make non-greasy sticks, such as deodorant sticks, but these sticks are often formulated with soaps, such as sodium stearate or triethanolamine stearate as a gelling agent, which contribute to a basic pH and may be aggressive on the skin.

Solid aqueous gels are also known, for example, in documents WO-A-97/17055 and WO-A-97/17053, the disclosures of which are incorporated by reference herein. However, these gels tend to use a fairly high concentration of gelling agent and tend to lack freshness and/or softness when applied to the skin. Such gels may also involve a special preparation technique, extrusion, which involves hydration of the gels at the time of use. Furthermore, it is not uncommon for these gels to become brittle over time.

Thus, there is a need for cosmetic compositions, such as solid aqueous cosmetic compositions, such as solid aqueous gel compositions, that help to alleviate at least one of the drawbacks mentioned above.

The inventor has discovered, unexpectedly, that cosmetic compositions, such as solid aqueous cosmetic compositions, for example, solid aqueous gel compositions, which may be obtained by a particular process, comprising at least one polyvinyl alcohol are suitable for use as cosmetic compositions.

One subject of the present invention is a solid aqueous cosmetic composition, for example, a solid aqueous gel, comprising, in a cosmetically acceptable medium, at least one polyvinyl alcohol comprising polymeric units of formula (I):

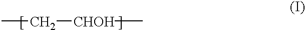

and optionally comprising at least one polymeric unit of formula (II):

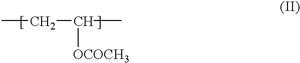

wherein said at least one polymeric unit of formula (II) is present in said at least one polyvinyl alcohol in a maximum amount of 5 mol % relative to said at least one polyvinyl alcohol,

and wherein said composition has a stress measurement ranging, for example, from 200 to 10,000 Pascals (N/m²), such as, for example, from 500 to 5,000 Pascals (Pa), and further such as, for example, from 1,000 to 4,000 Pa.

Another subject of the invention is a process for manufacturing a solid aqueous cosmetic composition, such as a solid aqueous gel, as defined above comprising (1) cooling a homogeneous aqueous solution of said at least one polyvinyl alcohol to a temperature of less than or equal to −5° C. and (2) heating said solution to a temperature ranging, for example, from 20° C. to 25° C.

Another subject of the invention is a cosmetic treatment process for a support chosen from, for example, facial skin, body skin, mucous membranes, and keratin fibers comprising applying a composition as defined below or a composition obtained by the process defined below to said support.

Another subject of the invention is a solid emulsion composition, an oil-in-water dispersion composition, a multi-phase system composition, a stick composition, or a tube composition comprising, in a cosmetically acceptable medium, at least one polyvinyl alcohol comprising polymeric units of formula (I):

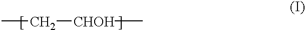

and optionally comprising at least one polymeric unit of formula (II):

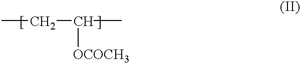

wherein said at least one polymeric unit of formula (II) is present in said at least one polyvinyl alcohol in a maximum amount of 5 mol % relative to said at least one polyvinyl alcohol,

and wherein said composition has a stress measurement ranging from 200 to 10,000 Pascals (N/m²).

Another subject of the invention is a body hygiene composition, a hair composition, a make-up composition for facial or body skin or for mucous membranes, a care composition for the skin or mucous membranes, an antisun composition or a self-tanning composition comprising a solid aqueous cosmetic composition which comprises, in a cosmetically acceptable medium, at least one polyvinyl alcohol comprising polymeric units of formula (I):

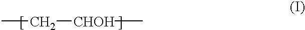

and optionally comprising at least one polymeric unit of formula (II):

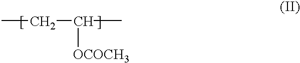

wherein said at least one polymeric unit of formula (II) is present in said at least one polyvinyl alcohol in a maximum amount of 5 mol % relative to said at least one polyvinyl alcohol,

and wherein said composition has a stress measurement ranging from 200 to 10,000 Pascals (N/m²).

Yet another subject of the invention is a lipstick, a stick foundation, a foundation cast in a dish, a face powder, an eyeshadow, a lipstick-fixing base, a concealer stick, a lip gloss, an eyeliner, a mascara, or a temporary tattoo product comprising, in a cosmetically acceptable medium, a solid aqueous cosmetic composition comprising at least one polyvinyl alcohol comprising polymeric units of formula (I):

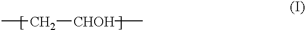

and optionally comprising at least one polymeric unit of formula (II):

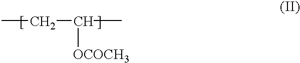

wherein said at least one polymeric unit of formula (II) is present in said at least one polyvinyl alcohol in a maximum amount of 5 mol % relative to said at least one polyvinyl alcohol,

and wherein said lipstick, stick foundation, foundation cast in a dish, face powder, eyeshadow, lipstick-fixing base, concealer stick, lip gloss, eyeliner, mascara, or temporary tattoo product has a stress measurement ranging from 200 to 10,000 Pascals (N/m²).

Another subject of the invention is a lipcare base, a body ointment, or a care stick comprising, in a cosmetically acceptable medium, at least one polyvinyl alcohol comprising polymeric units of formula (I):

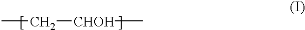

and optionally comprising at least one polymeric unit of formula (II):

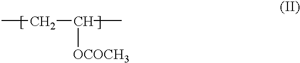

wherein said at least one polymeric unit of formula (II) is present in said at least one polyvinyl alcohol in a maximum amount of 5 mol % relative to said at least one polyvinyl alcohol,

and wherein said lipcare base, body ointment, or care stick has a stress measurement ranging from 200 to 10,000 Pascals (N/m²).

Another subject of the invention is a process for manufacturing at least one solid aqueous cosmetic composition comprising at least one cooling/heating cycle, which comprises: (1) cooling a homogeneous aqueous solution of at least one polyvinyl alcohol to a temperature of less than or equal to −5° C. and (2) heating said solution to a temperature ranging from 20-25° C., wherein said at least one solid aqueous cosmetic composition comprises, in a cosmetically acceptable medium, at least one polyvinyl alcohol comprising polymeric units of formula (I):

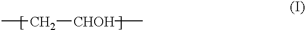

and optionally comprising at least one polymeric unit of formula (II):

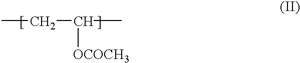

wherein said at least one polymeric unit of formula (II) is present in said at least one polyvinyl alcohol in a maximum amount of 5 mol % relative to said at least one polyvinyl alcohol,

and wherein said composition has a stress measurement ranging from 200 to 10,000 Pascals (N/m²).

Yet another subject of the invention is a cosmetic treatment process for at least one support chosen from facial skin, body skin, mucous membranes, and keratin fibers comprising applying to said at least one support a solid aqueous cosmetic composition comprising, in a cosmetically acceptable medium, at least one polyvinyl alcohol comprising polymeric units of formula (I):

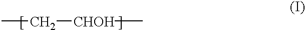

and optionally comprising at least one polymeric unit of formula (II):

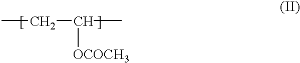

wherein said at least one polymeric unit of formula (II) is present in said at least one polyvinyl alcohol in a maximum amount of 5 mol % relative to said at least one polyvinyl alcohol,

and wherein said composition has a stress measurement ranging from 200 to 10,000 Pascals (N/m²).

Another subject of the invention is a cosmetic treatment process for at least one support chosen from facial skin, body skin, mucous membranes, and keratin fibers comprising applying to said support a solid aqueous cosmetic composition obtained by a process comprising (1) cooling a homogeneous aqueous solution of at least one polyvinyl alcohol to a temperature of less than or equal to −5° C. and (2) heating said solution to a temperature ranging from 20-25° C.

A further subject of the invention is a solid aqueous gel comprising, in a cosmetically acceptable medium, at least one polyvinyl alcohol comprising polymeric units of formula (I):

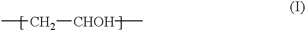

and optionally comprising at least one polymeric unit of formula (II):

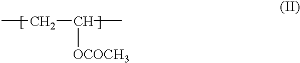

wherein said at least one polymeric unit of formula (II) is present in said at least one polyvinyl alcohol a maximum amount of 5 mol % relative to said at least one polyvinyl alcohol,

and wherein said gel has a stress measurement ranging from 200 to 10,000 Pascals (N/m²).

It is known practice, for example, from patents GB-A-1 342 152 and EP-A-0 107 055, the disclosures of which are incorporated by reference herein, to prepare polyvinyl alcohol hydrogels by freezing and then thawing an aqueous solution of polyvinyl alcohol. Such processes allow the preparation of fish baits or medical prostheses.

However, by selecting a specific type of polyvinyl alcohol, the inventor has discovered solid aqueous compositions, such as, for example, solid aqueous gels, with particular rheological properties making such compositions suitable for use in the field of cosmetics.

The compositions according to the invention tend to exhibit at least one stability characteristic chosen from time stability and heat stability. Further, the compositions according to the invention may also exhibit both time-stability and heat-stability.

Time stability is evaluated after storing at room temperature for at least one month, such as, for example, at least two months, the compositions of the invention. The compositions of the invention are considered to have time stability if the compositions, when so evaluated, show reduced or no syneresis (exudation) or phase separation, and the hardness of the compositions shows resistance to change. Heat stability is evaluated after storing at 45° C. for at least one month, such as, for example, at least two months, the compositions of the invention. The compositions of the invention are considered to have heat stability if the compositions, when so evaluated, show reduced or no syneresis (exudation) or phase separation, and the hardness of the compositions shows resistance to change.

The compositions according to the invention exhibit at least one desirable cosmetic property chosen from lack of stickiness when applied and user-friendliness.

Moreover, such compositions can be simple to prepare, such as, for example, by simple mixing of the constituents.

In one aspect, solid aqueous compositions according to the invention comprise, in a cosmetically acceptable medium, at least one polyvinyl alcohol comprising polymeric units of formula (I):

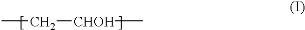

and optionally comprising at least one polymeric unit of formula (II):

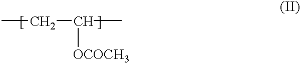

wherein said at least one polymeric unit of formula (II) is present in said at least one polyvinyl alcohol in a maximum amount of 5 mol % relative to said at least one polyvinyl alcohol

and wherein said composition has a stress measurement ranging, for example, from 200 to 10,000 Pascals (N/m²), such as, for example, from 500 to 5,000 Pa, and further such as, for example, from 1,000 to 4,000 Pa.

For example, the at least one unit of formula (II) can be present in said at least one polyvinyl alcohol in an amount ranging from 0 to 3 mol % relative to said at least one polyvinyl alcohol, such as, for example, from 0.05 to 2 mol % relative to said at least one polyvinyl alcohol.

An amount of 5 mol % relative to said at least one polyvinyl alcohol of the at least one unit of formula (II) corresponds to a degree of hydrolysis of 95%.

Said at least one polyvinyl alcohol can have a mass-average molecular weight (Mw) ranging from, for example, 10,000 g/mol to 1,000,000 g/mol, such as, for example, from 12,000 g/mol to 500,000 g/mol, and further such as, for example, from 15,000 g/mol to 200,000 g/mol.

Representatives of the at least one polyvinyl alcohol that may be used according to the invention can include, for example, the products sold under the names Rhodoviol 4-20, Rhodoviol 8-20, and Rhodoviol 30-5 by Rhodia, and Mowiol 4-98, Mowiol 10-98, and Mowiol 20-98 by Clariant.

The at least one polyvinyl alcohol may be present in the composition in an amount ranging, for example, from 1% to 50% by weight relative to the total weight of the composition, such as, for example, from 5% to 25% by weight relative to the total weight of the composition, and further such as, for example, from 7% to 20% by weight relative to the total weight of the composition.

The composition moreover comprises an aqueous phase, which may comprise at least one water chosen from floral waters such as cornflower water, mineral water such as eau de Vittel, eau de Lucas, and eau de la Roche Posay, and thermal spring water.

Water can be present in the composition according to the invention in an amount ranging, for example, from 50% to 99% by weight relative to the total weight of the composition, such as, for example, from 60% to 90% by weight relative to the total weight of the composition.

The composition according to the invention comprises a cosmetically acceptable medium, i.e., a medium that is compatible with use on keratin materials such as the skin, the nails, the hair, the eyelashes, the eyebrows, mucous membranes and semi-mucous membranes, and any other area of facial or body skin. This cosmetically acceptable medium may comprise, besides water and polyvinyl alcohol, the constituents usually used in the type of application envisaged.

Thus, in one embodiment, the composition according to the invention can additionally comprise at least one polyhydric alcohol (polyol), such as at least one polyol of low mass-average molecular weight, such as, for example, a molecular weight (Mw) of less than or equal to 500 g/mol. The at least one polyol can be chosen, for example, from polyhydric alcohols comprising from 1 to 18 carbon atoms and from 2 to 6 hydroxyl functions, such as polyhydric alcohols comprising from 2 to 12 carbon atoms and from 2 to 4 hydroxyl functions.

The at least one polyhydric alcohol can be chosen from, for example, ethylene glycol, isoprene glycol, glycerol, 1,2-propanediol (propylene glycol), dipropylene glycol, diethylene glycol, butylene glycol, pentylene glycol, hexylene glycol, diglycerol, erythritol, arabitol, adonitol, sorbitol, and dulcitol.

The at least one polyhydric alcohol according to the invention may be present in an amount ranging, for example, from 0% to 20% by weight relative to the total weight of the composition, such as, for example, from 0.05% to 15% by weight relative to the total weight of the composition, and further such as, for example, from 0.1% to 10% by weight relative to the total weight of the composition.

The composition may also comprise at least one poly(C₂-C₅ oxyalkylene), such as at least one poly(C₂-C₅ oxyalkylene) chosen from, for example, poly(ethylene oxide) and poly(propylene oxide).

The at least one poly(C₂-C₅ oxyalkylene) can have a mass-average molecular weight, for example, ranging from 1,000 g/mol to 6,000 g/mol, such as from 1,500 g/mol to 5,000 g/mol.

The at least one poly(C₂-C₅ oxyalkylene) may be present in the composition according to the invention in an amount ranging, for example, from 0% to 10% by weight relative to the total weight of the composition, such as, for example, from 1% to 8% by weight relative to the total weight of the composition, and further such as, for example, from 2% to 5% by weight relative to the total weight of the composition.

The composition according to the invention may also comprise at least one solvent other than water, such as, for example, C₁-C₆ alcohols such as ethanol and isopropanol.

The composition according to the invention may also comprise water-soluble compounds, such as, for example, water-soluble compounds chosen from water-soluble dyes, water-soluble cosmetic active agents, and water-soluble pharmaceutical active agents.

Representative water-soluble dyes can include, for example, the disodium salt of ponceau, the disodium salt of alizarin green, quinoline yellow, the trisodium salt of amaranth, the disodium salt of tartrazine, the monosodium salt of rhodamine, the disodium salt of fuchsin, and xanthophyll.

Representative water-soluble active agents can include, for example, water-soluble vitamins, water-soluble sunscreens, active principles such as hyaluronic acid and its derivatives, and magnesium gluconate, trace elements, amino acids, antimicrobial agents, and cosmetic active agents that are commonly used.

The composition according to the invention may also comprise at least one additional ingredient, such as, for example, additional ingredients chosen from pigments, nacres, and fillers.

The term “pigment” should be understood as meaning white or colored, mineral or organic particles which are insoluble in the medium and which are intended to color and/or opacify the composition. The term “nacre” should be understood as meaning iridescent particles which reflect light. The term “filler” should be understood as meaning colorless or white, mineral or synthetic, lamellar or non-lamellar particles which are intended to give body or rigidity to the composition, and/or softness, a matt effect and uniformity to the make-up.

The pigments may be present in an amount ranging, for example, from 0% to 40% by weight relative to the total weight of the gel, such as, for example, from 0.1% to 30% by weight relative to the total weight of the gel, and further such as, for example, from 1% to 20% by weight relative to the total weight of the gel. The pigments can be chosen from typical- and nanometric-sized white pigments, colored pigments, mineral pigments, and organic pigments. The expression “nanometric-sized” means pigments whose mean particle size ranges, for example, from 5 nm to 100 nm. Representative mineral pigments and nanopigments can include, for example, titanium dioxide, zirconium dioxide, and cerium dioxide, as well as zinc oxide, iron oxide, chromium oxide, nanotitanias, and ferric blue. Representative organic pigments can include, for example, carbon black and the lakes commonly used to give the lips and the skin a make-up effect, which are calcium, barium, aluminium, and zirconium salts of acidic dyes, such as halo-acid dyes, azo dyes, and anthraquinone dyes.

The nacres may be present in the composition in an amount ranging from, for example, from 0% to 40% by weight relative to the total weight of the composition, such as, for example, from 0.1% to 30% by weight relative to the total weight of the composition, and further such as, for example, from 1% to 20% by weight relative to the total weight of the composition. Representative nacres which may be used according to the invention can include, for example, natural mother-of-pearl, mica coated with titanium oxide, mica coated with iron oxide, mica coated with natural pigment, mica coated with bismuth oxychloride, and colored titanium mica.

The fillers may be present in the composition according to the invention in an amount ranging, for example, from 0% to 60% by weight relative to the total weight of the composition, such as, for example, from 0.1% to 40% by weight relative to the total weight of the composition, and further such as, for example, from 1% to 20% by weight relative to the total weight of the composition. Representative fillers can include, for example, talc, mica, silica, kaolin, Nylon (polyamide) powder, poly-β-alanine powder, polyethylene powder, Teflon (polytetrafluoroethylene), lauroyllysine, starch, boron nitride, bismuth oxychloride, tetrafluoroethylene polymer powders, polystyrene powders, polyester powders, hollow synthetic microspheres, microsponges, silicone resin microbeads, hollow silica microspheres, glass microcapsules, and ceramic microcapsules.

Moreover, the composition according to the invention may comprise a fatty phase (wherein, for example, the fatty phase is dispersed in a continuous aqueous phase). This fatty phase may comprise at least one additive chosen from oils, animal waxes, plant waxes, mineral waxes, synthetic waxes, and lipophilic cosmetic active agents.

The composition according to the invention can also comprise at least one additive chosen from additives commonly used in the field under consideration, such as, for example, the field of cosmetics. Representative at least one additives can include, for example, antioxidants, fragrances, essential oils, preserving agents, vitamins, sphingolipids, self-tanning compounds, such as DHA, sunscreens, and surfactants.

One skilled in the art should take care to select such optional additives, and/or the amount thereof, such that the advantageous properties of the composition according to the invention are not, or are not substantially, adversely affected by the addition envisaged.

The composition according to the invention can be in solid aqueous form. This means that no collapse of the composition is observed when it is out of the container comprising it, in the absence of a mechanical or thermal stimulation (in particular heating).

For the purposes of the present invention, the stress measurement is measured on a composition of the invention after storing the composition for at least two days at a temperature ranging, for example, from 20° C. to 25° C., such as at a temperature of 22° C., under a relative humidity of 50%. After penetration with a flat cylindrical stainless steel spindle 6 (or 12) mm in diameter, into the matrix of the composition to a depth of 0.4 mm at a speed of 1 mm/s, relaxation for 1 second and removal of the said spindle from the matrix of the composition at a speed of 1 mm/s, the stress is measured with a texture analyser of the “TA-XT2” type sold by the company Rheo. In general, the stress measurement of such compositions tends to become constant at about 5 days. Compositions of the invention have a stress measurement ranging, for example, from 200 to 10,000 Pascals (N/m²).

The compositions according to the invention can have a stress measurement ranging, for example, from 500 to 5,000 Pa, such as from 1,000 to 4,000 Pa.

The compositions can also have a storage modulus ranging, for example, from 500 Pa to 10,000 Pa, such as for example from 1,000 Pa to 5,000 Pa.

The compositions can also have a loss modulus ranging, for example, from 50 Pa to 1,000 Pa, such as from 150 Pa to 500 Pa.

The method for measuring these moduli is described before the examples.

Using these moduli, it is possible to calculate the complex modulus G* which is equal to the square root of the sum of the squares of the storage modulus and loss modulus.

The modulus G* of the compositions according to the invention can range, for example, from 200 to 10,000 Pa, such as, for example, from 1,000 to 4,000 Pa.

The compositions according to the invention can be prepared, for example, by a process comprising (1) cooling a homogeneous aqueous solution of at least one polyvinyl alcohol to a temperature of less than or equal to −5° C., and (2) heating said homogeneous aqueous solution to room temperature (20° C. to 25° C.).

The starting homogeneous aqueous solution may be obtained by any means. The expression “homogeneous solution” means a solution in which the chains of the at least one polyvinyl alcohol are totally hydrated, which leads to a totally transparent solution. This is understood in terms of the conventional definition given in the dictionary. Thus, a transparent composition allows light to pass through easily and makes it possible clearly to distinguish objects through its thickness. In general, a transparent composition will have a maximum light transmittance value, irrespective of the wavelength ranging from 400 nm to 800 nm, through a 1 cm thick sample, of at least 35%, such as, for example, of at least 50% (see EP-A-0 291 334, the disclosure of which is incorporated by reference herein). The transmittance may be measured by placing a 1 cm thick sample in the light beam of a spectrophotometer working in the wavelengths of the light spectrum.

It is possible to prepare the said homogeneous solution by heating a water/polyvinyl alcohol mixture to a temperature of about 80-90° C.

The homogeneous solution thus obtained can then be cooled immediately to −5° C. or below, or may be left to cool to room temperature (20-25° C.) before carrying out the cooling step down to −5° C. or below. This cooling step may be carried out by any common method such as using a bath of ice-cold water, liquid nitrogen or a freezer. The speed at which the cooling step down to −5° C. or below is carried out may range, for example, from 0.1° C. to 1° C. per minute such as from 0.3 to 0.7° C. per minute.

In one embodiment of the invention, the homogeneous solution of polyvinyl alcohol can be introduced into a mold before being cooled to the desired temperature.

The temperature to which the solution is cooled is less than or equal to −5° C., such as, for example, ranging from −50° C. to −10° C., and further such as, for example, from 30° C. to −15° C. It has been found that this temperature has an influence on the elasticity and flexibility of the gel subsequently obtained.

This is likewise the case for the duration for which the solution is maintained at this temperature. This duration may range from, for example, 5 to 48 hours, such as, for example, from 10 to 30 hours.

The cooled or frozen solution is then heated by any common means such as an oil bath, up to a temperature ranging from, for example, 20° C. to 25° C., such as for example from 22° C. to 23° C.

The speed at which the heating step is carried out may range, for example, from 0.1° C. to 1° C. per minute, such as from 0.2° C. to 0.5° C. per minute.

The composition may then be used in its current form or it may be subjected to at least one other cooling/heating cycle, also known as freezing/thawing cycles.

Specifically, it has been found that the number of freezing/thawing cycles can influence the hardness of the compositions, and also their stability.

Thus, the composition obtained may be left after the first cycle, for a time ranging, for example, from 10 minutes to 24 hours, such as from, for example, 30 minutes to 12 hours at room temperature; next, it may be cooled again in the manner and under the conditions described above, before heating it in the manner and under the conditions described above. For example, two, three, four, five, or six freezing/thawing cycles may thus be carried out. In some embodiments, for example, two, three or four freezing/thawing cycles are carried out.

An aqueous composition according to the invention may be in any cosmetically acceptable form which may be envisaged, such as a solid aqueous gel; a solid emulsion generally of oil-in-water or multiple type; an oil-in-water dispersion; a multi-phase system such as for example a two-phase system. The composition in one embodiment, for example, may be in the form of a stick or tube.

This composition can be employed as a make-up or care composition for the skin, such as, for example, for the body, the face and/or the scalp, or for keratin fibres, such as, for example, the hair, the nails, the eyelashes and/or the eyebrows, or alternatively for mucous membranes, such as, for example, the lips. It may be in the form of a body hygiene composition, such as, for example, in the form of a deodorant stick; a hair composition, such as, for example, a styling stick or make-up stick for the hair; a make-up composition for facial or body skin or for mucous membranes, such as, for example, a lipstick, a foundation cast as a stick or a dish, a face powder, an eyeshadow, a fixing base to be applied over a standard lipstick, a concealer stick, a lip gloss, an eyeliner, a mascara or temporary tattoo products; a care composition for the skin or mucous membranes, such as, for example, a lipcare base, a body ointment or a care stick; an antisun composition or a self-tanning composition.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The invention is illustrated in greater detail in the examples which follow without however exhibiting a limiting character.

Methods For Measuring The Storage Modulus And Loss Modulus

The apparatus used in the examles was a Haake RS 150 rheometer in dynamic mode. The rheological measurements were performed at 25° C. A sinusoidal stress sweep was carried out while remaining in the domain of linear viscoelasticity. The frequency was set at a constant 1 hertz. The stress magnitude was increased from 0.1 Pa to 1000 Pa logarithmically. Measurements were taken six times for every ten-fold increase in stress magnitude.

The geometry used corresponds to a cone-plane 6 cm (or 3.5 cm) in diameter, forming an angle of 2 °with the plane.

The storage modulus and loss modulus were determined from the deformation of the gel subjected to a sinusoidal stress. The standard dynamic rheology equations describe the procedure which is followed and can be found in a book by P. Sherman, Industrial Rheology Ch. 1 (1970), the disclosure of which is incorporated by reference herein, and that also references storage modulus and loss modulus measurements. See Industrial Rheology Ch. 1, pp. 22-25. The complex modulus (G*) was calculated from the storage modulus (G′) and the loss modulus (G″). See Industrial Rheology Ch. 1, p. 23-24. The value of the complex modulus was calculated from the linear viscoelastic region where the storage modulus and the loss modulus are independent of the stress amplitude. The storage modulus corresponds to the energy stored by the material during the deformation, while the loss modulus corresponds to the energy dissipated.

### EXAMPLE 1

### Preparation of a Homogeneous Aqueous Solution of Polyvinyl Alcohol

An aqueous solution of polyvinyl alcohol with a mass-average molecular weight of 22,000 g/mol and a degree of hydrolysis ranging from 97.5% to 99.5% was prepared by mixing 15 g of polyvinyl alcohol in 85 ml of water and heating at 85° C. for 3 hours with stirring. After cooling, a homogeneous aqueous solution (at 15%) was obtained, which served as base for the following examples.

### Preparation of the Gels

The above solution was poured into three different bottles, in equal amount.

The first solution (S1) was cooled to −20° C. at a speed of 0.5° C./min, maintained at −20° C. for 23 hours and heated to 20° C. at a speed of 0.3° C./min. This corresponds to a single freezing/thawing cycle. A gel G1 was obtained.

The second solution (S2) was cooled to −20° C. at a speed of 0.5° C./min, maintained at −20° C. for 23 hours and heated to 22° C. at a speed of 0.3° C./min; it was maintained at 22° C. for 1 hour and then cooled again to −20° C. at a speed of 0.5° C./min, maintained at −20° C. for 23 hours and heated to 20° C. at a speed of 0.3° C./min. This corresponds to two freezing/thawing cycles. A gel G2 is obtained.

The third solution (S3) was cooled to −20° C. at a speed of 0.5° C./min, maintained at −20° C. for 23 hours and heated to 22° C. at a speed of 0.3° C./min; it was maintained at 22° C. for 1 hour and then cooled again to −20° C. at a speed of 0.5° C./min, maintained at −20° C. for 23 hours and heated to 22° C. at a speed of 0.3° C./min; it was maintained at 22° C. for 1 hour and then cooled again to −20° C. at a speed of 0.5° C./min, maintained at −20° C. for 23 hours and heated to 20° C. at a speed of 0.3° C./min. This corresponds to three freezing/thawing cycles. A gel G3 was obtained.

The stress of the gels obtained was measured (in Pa) after storing for several days at 22° C. under a relative humidity of 50%.

The following results were obtained:

| | After | After | After | After | After |
|---|---|---|---|---|---|
| | 2 days | 6 days | 9 days | 16 days | 32 days |
| Gel G1 | 310 | 400 | 390 | 510 | 810 |
| (1 cycle) | | | | | |
| Gel G2 | 980 | 1110 | 1290 | 1680 | 1710 |
| (2 cycles) | | | | | |
| Gel G3 | 1280 | 1420 | 1620 | 1650 | 1780 |
| (3 cycles) | | | | | |

It is found that the gelation phenomenon continues gradually over time.

### EXAMPLE 2

The above three gels were prepared again, along with a fourth gel G4 obtained after 4 freezing/thawing cycles.

The storage modulus and loss modulus were measured and the complex modulus G* was calculated (in Pa).

The following results were obtained:

| | After 3 days | After 6 days | After 12 days |
|---|---|---|---|
| Gel G1 (1 cycle) | 1050 | 1320 | 1710 |
| Gel G2 (2 cycels) | 2150 | 2750 | 2570 |
| Gel G3 (3 cycles) | 2110 | 2685 | 3980 |
| Gel G4 (4 cycles) | 2665 | 2632 | 3500 |

### EXAMPLE 3

Two solutions of polyvinyl alcohol at 15% in water were prepared, according to Example 1, and were subjected to 1, 2 or 3 cycles.

The following results were obtained:

| | After 1 | After 2 | After 3 |
|---|---|---|---|
| | cycle | cycles | cycles |
| PVA of mass-average molecular | Gel | Gel | Gel |
| weight 22,000 g/mol and degree of | | | |
| hydrolysis of 97.5-99.5% | | | |
| PVA of mass-average molecular | No gel | No gel | No gel |
| weight 15 000 g/mol and degree of | | | |
| hydrolysis of 86-89% | | | |

It is thus found that when the degree of hydrolysis is too low, it is not possible to obtain an adequate gel.

### EXAMPLE 4

A colored solution was prepared comprising:

10 g of polyvinyl alcohol of MW=22 000 and degree of hydrolysis: 97.5-99.5%

5 g of water-soluble dye

qs 100 ml of water.

The solution was cooled to −20° C. at a speed of 0.5° C./min, maintained at −20° C. for 23 hours and heated to 22° C. at a speed of 0.3° C./min; it was then cooled again to −20° C. at a speed of 0.5° C./min, maintained at −20° C. for 23 hours and heated to 20° C. at a speed of 0.3° C./min.

A colored stick which may be used in cosmetics was obtained.

What is claimed is:

1. A solid aqueous cosmetic composition comprising, in a cosmetically acceptable medium, at least one polyvinyl alcohol comprising polymeric units of formula (I): 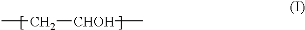
and optionally comprising at least one polymeric unit of formula (II): 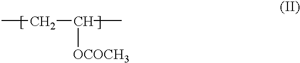
wherein said at least one polymeric unit of formula (II) is present in said at least one polyvinyl alcohol in a maximum amount of 5 mol % relative to said at least one polyvinyl alcohol,
and wherein said composition has a stress measurement ranging from 200 to 10,000 Pascals (N/m²).

2. A composition according to claim 1, wherein said stress measurement ranges from 500 to 5,000 Pa.

3. A composition according to claim 1, wherein said stress measurement ranges from 1,000 to 4,000 Pa.

4. A composition according to claim 1, wherein said composition has at least one property chosen from a storage modulus ranging from 500 Pa to 10,000 Pa, a loss modulus ranging from 50 Pa to 1,000 Pa, and a complex modulus G* ranging from 200 to 10,000 Pa.

5. A composition according to claim 4, wherein said storage modulus ranges from 1,000 Pa to 5,000 Pa.

6. A composition according to claim 4, wherein said loss modulus ranges from 150 Pa to 500 Pa.

7. A composition according to claim 4, wherein said complex modulus G* ranges from 1,000 to 4,000 Pa.

8. A composition according to claim 1, wherein said at least one unit of formula (II) is present in said at least one polyvinyl alcohol in an amount ranging from 0 to 3 mol % relative to said at least one polyvinyl alcohol.

9. A composition according to claim 8, wherein said at least one unit of formula (II) is present in said at least one polyvinyl alcohol in an amount ranging from 0.05 to 2 mol % relative to said at least one polyvinyl alcohol.

10. A composition according to claim 1, wherein said at least one polyvinyl alcohol has a mass-average molecular weight (Mw) ranging from 10,000 g/mol to 1,000,000 g/mol.

11. A composition according to claim 10, wherein said at least one polyvinyl alcohol has a mass-average molecular weight (Mw) ranging from 12,000 g/mol to 500,000 g/mol.

12. A composition according to claim 11, wherein said at least one polyvinyl alcohol has a mass-average molecular weight (Mw) ranging from 15,000 g/mol to 200,000 g/mol.

13. A composition according to claim 1, wherein said at least one polyvinyl alcohol is present in an amount ranging from 1% to 50% by weight relative to the total weight of the composition.

14. A composition according to claim 13, wherein said at least one polyvinyl alcohol is present in an amount ranging from 5% to 25% by weight relative to the total weight of the composition.

15. A composition according to claim 14, wherein said at least one polyvinyl alcohol is present in an amount ranging from 7% to 20% by weight relative to the total weight of the composition.

16. A composition according to claim 1, further comprising at least one polyhydric alcohol.

17. A composition according to claim 16, wherein said at least one polyhydric alcohol is chosen from polyhydric alcohols with a mass-average molecular weight (Mw) of less than or equal to 500 g/mol.

18. A composition according to claim 16, wherein said at least one polyhydric alcohol is chosen from polyhydric alcohols comprising from 1 to 18 carbon atoms and from 2 to 6 hydroxyl functions.

19. A composition according to claim 18, wherein said at least one polyhydric alcohol is chosen from polyhydric alcohols comprising from 2 to 12 carbon atoms and from 2 to 4 hydroxyl functions.

20. A composition according to claim 16, wherein said at least one polyhydric alcohol is present in an amount ranging from 0% to 20% by weight relative to the total weight of the composition.

21. A composition according to claim 20, wherein said at least one polyhydric alcohol is present in an amount ranging from 0.05% to 15% by weight relative to the total weight of the composition.

22. A composition according to claim 21, wherein said at least one polyhydric alcohol is present in an amount ranging from 0.1% to 10% by weight relative to the total weight of the composition.

23. A composition according to claim 1, further comprising at least one poly(C₂-C₅ oxyalkylene).

24. A composition according to claim 23, wherein said at least one poly(C₂-C₅ oxyalkylene) is chosen from poly(ethylene oxide) and poly(propylene oxide).

25. A composition according to claim 23, wherein said at least one poly(C₂-C₅ oxyalkylene) is present in an amount ranging from 0% to 10% by weight relative to the total weight of the composition.

26. A composition according to claim 25, wherein said at least one poly(C₂-C₅ oxyalkylene) is present in an amount ranging from 1% to 8% by weight relative to the total weight of the composition.

27. A composition according to claim 26, wherein said at least one poly(C₂-C₅ oxyalkylene) is present in an amount ranging from 2% to 5% by weight relative to the total weight of the composition.

28. A composition according to claim 1, further comprising at least one water-soluble compound.

29. A composition according to claim 28, wherein said at least one water-soluble compound is chosen from water-soluble dyes, water-soluble cosmetic active agents, and water-soluble pharmaceutical active agents.

30. A composition according to claim 1, further comprising at least one additive chosen from pigments, nacres, and fillers.

31. A solid emulsion composition, an oil-in-water dispersion composition, a multi-phase system composition, a stick composition, or a tube composition comprising, in a cosmetically acceptable medium, at least one polyvinyl alcohol comprising polymeric units of formula (I): 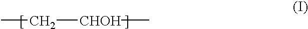
and optionally comprising at least one polymeric unit of formula (II): 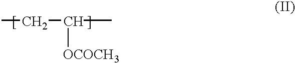
wherein said at least one polymeric unit of formula (II) is present in said at least one polyvinyl alcohol in a maximum amount of 5 mol % relative to said at least one polyvinyl alcohol,
and wherein said composition has a stress measurement ranging from 200 to 10,000 Pascals (N/m²).

32. A composition according to claim 31, wherein said solid emulsion compositions are chosen from oil-in-water solid emulsion compositions and multiple solid emulsion compositions.

33. A composition according to claim 31, wherein said multi-phase system is a two-phase system.

34. A body hygiene composition, a hair composition, a make-up composition for facial or body skin or for mucous membranes, a care composition for the skin or mucous membranes, an antisun composition or a self-tanning composition comprising a solid aqueous cosmetic composition which comprises, in a cosmetically acceptable medium, at least one polyvinyl alcohol comprising polymeric units of formula (I): 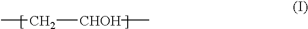
and optionally comprising at least one polymeric unit of formula (II): 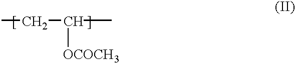
wherein said at least one polymeric unit of formula (II) is present in said at least one polyvinyl alcohol in a maximum amount of 5 mol % relative to said at least one polyvinyl alcohol,
and wherein said composition has a stress measurement ranging from 200 to 10,000 Pascals (N/m²).

35. A composition according to claim 34, wherein said body hygiene composition is a deodorant stick.

36. A composition according to claim 34, wherein said hair composition is chosen from styling sticks for the hair and make-up sticks for the hair.

37. A lipstick, a stick foundation, a foundation cast in a dish, a face powder, an eyeshadow, a lipstick-fixing base, a concealer stick, a lip gloss, an eyeliner, a mascara, or a temporary tattoo product comprising, in a cosmetically acceptable medium, a solid aqueous cosmetic composition comprising at least one polyvinyl alcohol comprising polymeric units of formula (I): 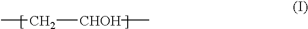
and optionally comprising at least one polymeric unit of formula (II): 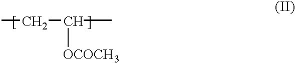
wherein said at least one polymeric unit of formula (II) is present in said at least one polyvinyl alcohol in a maximum amount of 5 mol % relative to said at least one polyvinyl alcohol,
and wherein said lipstick, stick foundation, foundation cast in a dish, face powder, eyeshadow, lipstick-fixing base, concealer stick, lip gloss, eyeliner, mascara, or temporary tattoo product has a stress measurement ranging from 200 to 10,000 Pascals (N/m²).

38. A lipcare base, a body ointment, or a care stick comprising, in a cosmetically acceptable medium, at least one polyvinyl alcohol comprising polymeric units of formula (I): 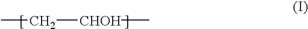
and optionally comprising at least one polymeric unit of formula (II): 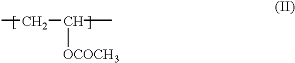
wherein said at least one polymeric unit of formula (II) is present in said at least one polyvinyl alcohol in a maximum amount of 5 mol % relative to said at least one polyvinyl alcohol,
and wherein said lipcare base, body ointment, or care stick has a stress measurement ranging from 200 to 10,000 Pascals (N/m²).

39. A process for manufacturing at least one solid aqueous cosmetic composition comprising at least one cooling/heating cycle, which comprises: (1) cooling a homogeneous aqueous solution of at least one polyvinyl alcohol to a temperature of less than or equal to −5° C. and (2) heating said solution to a temperature ranging from 20-25° C., wherein said at least one solid aqueous cosmetic composition comprises at least one polyvinyl alcohol comprising polymeric units of formula (I): 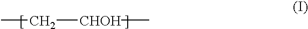
and optionally comprising at least one polymeric unit of formula (II): 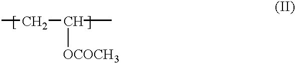
wherein said at least one polymeric unit of formula (II) is present in said at least one polyvinyl alcohol in a maximum amount of 5 mol % relative to said at least one polyvinyl alcohol,
and wherein said composition has a stress measurement ranging from 200 to 10,000 Pascals (N/m²).

40. A process according to claim 39, wherein said solution is cooled to a temperature ranging from −50° C. to −10° C.

41. A process according to claim 40, wherein said solution is cooled to a temperature ranging from −30° C. to −15° C.

42. A process according to claim 39, wherein said solution is cooled at a rate ranging from 0.1-1° C. per minute.

43. A process according to claim 42, wherein said solution is cooled at a rate ranging from 0.3-0.7° C. per minute.

44. A process according to claim 39, further comprising, after said cooling and before said heating, maintaining said solution at a temperature of less than or equal to −5° C. for a time ranging from 5 to 48 hours.

45. A process according to claim 44, further comprising, after said cooling and before said heating, maintaining said solution at a temperature of less than or equal to −5° C. for a time ranging from 10 to 30 hours.

46. A process according to claim 39, wherein said solution is heated to a temperature of 22-23° C.

47. A process according to claim 39, wherein said solution is heated at a rate ranging from 0.1-1° C. per minute.

48. A process according to claim 47, wherein said solution is heated at a rate ranging from 0.2-0.5° C. per minute.

49. A process according to claim 39, wherein said cooling/heating cycle is carried out up to a total of six times.

50. A process according to claim 49, wherein said cooling/heating cycle is carried out up to a total of four times.

51. A cosmetic treatment process for at least one support chosen from facial skin, body skin, mucous membranes, and keratin fibers comprising applying to said at least one support a solid aqueous cosmetic composition comprising, in a cosmetically acceptable medium, at least one polyvinyl alcohol comprising polymeric units of formula (I): 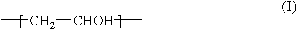
and optionally comprising at least one polymeric unit of formula (II): 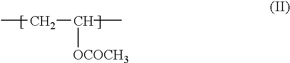
wherein said at least one polymeric unit of formula (II) is present in said at least one polyvinyl alcohol in a maximum amount of 5 mol % relative to said at least one polyvinyl alcohol,
and wherein said composition has a stress measurement ranging from 200 to 10,000 Pascals (N/m²).

52. A cosmetic treatment process for at least one support chosen from facial skin, body skin, mucous membranes, and keratin fibers comprising applying to said support a solid aqueous cosmetic composition obtained by a process comprising (1) cooling a homogeneous aqueous solution of at least one polyvinyl alcohol to a temperature of less than or equal to −5° C. and (2) heating said solution to a temperature ranging from 20-25° C.

53. A solid aqueous gel comprising, in a cosmetically acceptable medium, at least one polyvinyl alcohol comprising polymeric units of formula (I): 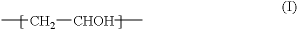
and optionally comprising at least one polymeric unit of formula (II): 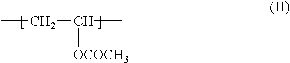
wherein said at least one polymeric unit of formula (II) is present in said at least one polyvinyl alcohol a maximum amount of 5 mol % relative to said at least one polyvinyl alcohol,
and wherein said gel has a stress measurement ranging from 200 to 10,000 Pascals (N/m²).
